(19) United States
(12) Patent Application Publication
(10) Pub. No.: US 2005/0025729 A1
(43) Pub. Date: Feb. 3, 2005

(54) Lipstick

(75) Inventors: Birgit Groteluschen, Wildeshausen (DE); Melanie Kovacevic, Hamburg (DE); Ghita Lanzendorfer, Hamburg (DE); Anja Muller, Rumpel (DE); Cornelia Riedel, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/868,256

(22) Filed: Jun. 14, 2004

Related U.S. Application Data
(63) Continuation of application No. PCT/EP02/14137, filed on Dec. 12, 2002.

(30) Foreign Application Priority Data
Dec. 13, 2001 (DE) 101 61 170.6

(51) Int. Cl. A61K 7/021; A61K 7/13

(52) U.S. Cl. 424/63

(57) ABSTRACT

The invention is a cosmetic or dermatological formulation comprising a) a lipophilic phase in a concentration of 20 to 70% by weight, b) an aqueous phase in a concentration of 20 to 40% by weight, c) at least one Pickering pigment selected from the group consisting of titanium dioxide having a particle diameter of 10 to 100 nm and iron oxide having a particle diameter of 100 to 800 nm, in a concentration of 1 to 6% by weight, d) at least one coated titanium dioxide pigment having a particle diameter of 100 to 2000 nm, in a concentration of 0.1 to 10% by weight, and e) further colored pigments having a in an amount of 0.1 to 20% by weight. The invention also includes a method for manufacturing lipstick comprising combining the above ingredients and methods of protecting the lips and decorating the lips comprising applying the formulation.

### CROSS-REFERENCE TO RELATED APPLICATIONS

This is a continuation application of PCT/EP02/14137, filed Dec. 12, 2002, which is incorporated herein by reference in its entirety, and also claims the benefit of German Priority Application No. 101 61 170.6, filed Dec. 13, 2001.

### FIELD OF THE INVENTION

The present invention relates to cosmetic or dermatological formulations comprising a) a lipophilic phase in a concentration of 20 to 70% by weight, b) an aqueous phase in a concentration of 20 to 40% by weight, c) Pickering pigments from titanium dioxide having a particle diameter of 10 to 100 nm and/or iron oxides having a particle diameter of 100 to 800 nm in an amount of 1 to 6% by weight, d) coated titanium dioxide pigments having a particle diameter of 100 to 2,000 nm in an amount of 0.1 to 10% by weight, and e) further colored pigments in an amount of 0.1 to 20% by weight, in each case based on the total weight of the formulation, in addition to optionally further cosmetic and/or dermatological active compounds, auxiliaries and additives, and the use thereof.

### BACKGROUND OF THE INVENTION

The desire to look beautiful and attractive has been rooted in humans for thousands of years. Although the ideal beauty has undergone changes in the course of time, achievement of a flawless outer appearance has always been the aim of humans, since a sympathetic appearance increases their feeling of their own value and the attractiveness to their fellow humans. The term decorative cosmetics is derived from the Latin “decoratio”—the accentuation of beauty. In this context, individual parts of the body, especially on the face, are usually accentuated and colored irregularities attenuated with the aid of dyestuffs.

Lipstick is one of the most used decorative cosmetics. More than 50% of women in Germany use it. In addition to emphasizing the lips with color, it is usually also used for lip care. As well as conventional lipstick, lip gloss and contour sticks are also used. Lip gloss gives the lips an intense decorative gloss, contour sticks serve to emphasize the edges of the lips.

The skin of the lips comprises a thin horny layer. The corium underneath pushes well-circulated papillae to close below the surface of the lips. The natural appearance of the lips originates from this. No sweat glands are to be found on the lips. They are chiefly moistened by saliva. There are also only isolated sebaceous glands, so that the surface of the lips is practically fat-free. The lips therefore tend to dry out and become cracked, especially on cold and dry days [W. Umbach (ed.): Kosmetik, Entwicklung, Herstellung und Anwendung kosmetischer Mittel [Cosmetics, Development, Manufacture and Use of Cosmetic Agents] (2nd edition), Thieme-Verlag Stuttgart, 1995].

As a rule, lipsticks comprise a wax matrix, into which liquid and semi-solid oils as well as pigments and fillers are incorporated. These anhydrous systems have not hitherto allowed water-soluble constituents to be introduced into the formulations. To remedy this disadvantage of the prior art, there have been a large number of attempts to formulate stick-like W/O emulsions. As a rule, in these cases relatively large amounts of emulsifier must be employed in order to obtain both a stable emulsion and pigment dispersion (e.g. EP 1 018 332, EP 1 064 908).

It was thus the object of the present invention to formulate water-containing lipsticks into which surface-active pigments are incorporated as stabilizers of the water drops in the oily phase.

### SUMMARY OF THE INVENTION

Surprisingly, this object has been achieved by cosmetic or dermatological formulations comprising a) a lipophilic phase in a concentration of 20 to 70% by weight, b) an aqueous phase in a concentration of 20 to 40% by weight, c) Pickering pigments from titanium dioxide having a particle diameter of 10 to 100 nm and/or iron oxides having a particle diameter of 100 to 800 nm in an amount of 1 to 6% by weight, d) coated titanium dioxide pigments having a particle diameter of 100 to 2000 nm in an amount of 0.1 to 10% by weight, and e) further colored pigments in an amount of 0.1 to 20% by weight, in each case based on the total weight of the formulation, in addition to optionally further cosmetic or dermatological active compounds, auxiliaries and additives.

In Pickering emulsions, the solid substance (the Pickering pigment) is concentrated at the oil/water phase boundary in the form of a layer, as a result of which the disperse phases are prevented from merging. The surface properties of the solid particles, which should show both hydrophilic and lipophilic properties, in particular are of essential importance in this context.

EP 0686 391 indeed also describes emulsions with surface-active pigments as the surface-active agent, but it was not possible for this specification to point the way to the present invention, since polyalkylsilsesquioxane particles are employed in it as the Pickering pigment.

Emulsions with pigments as an emulsifier substitute are also indeed known from other specifications. However, in these boron nitride (DE 199 39 836), modified laminar silicates (DE 199 39 835, DE 199 34 012) and microfine polymer particles (DE 198 34 819) are employed as surface-active pigments.

The specifications DE 197 12 483, DE 198 26 118, 198 42 730 and 198 42 732 describe quite generally emulsifier-free Pickering emulsions with microfine surface-active particles. In some cases, however, metal oxides having a particle size of less than 200 nm are expressly disclosed in these. In spite of the extensive prior art, however, it was in no way obvious for the expert to arrive at the achievement according to the invention of the object. The peculiarity of the formulations according to the invention is the complex composition of various pigments. In addition to the surface-active pigments, coloring pigments are also absolutely essential in order to give the formulation the consistency and stability desired for lipsticks. DE 198 26 118 indeed discloses decorative lipstick formulations in the examples. However, these comprise Betone 38, which leads to formulations which are highly viscous in the melt and difficult to process. This viscosity problem also arises with most of the other formulations known to date with surface-active pigments, such as, e.g., in the case of boron nitride (DE 198 39 836) or DE 198 39 835. This disadvantage surprisingly no longer exists with the formulations according to the invention. Their melts are of low viscosity and can be processed without problems.

DE 43 03 983 describes light protection formulations, but no decorative lipsticks. The formulations disclosed in this specification comprise no colored pigments and do not have a consistency typical for lipsticks, so that it was not possible for this disclosure to point the way to the present invention.

Finally, DE 198 42 730 and DE 198 42 732 indeed in principle disclose Pickering emulsions which can also be used as make-up or lip care products, but the intrinsic peculiarities of lipsticks are not taken into account in these specifications. Lipsticks are highly complex cosmetic formulations, the sensory and physical properties of which (e.g. color, viscosity, creaminess, adhesion to the skin) arise only from the interaction of the individual components with one another in a manner which is not obvious even for the expert. The fact that the achievement of the object of the invention is rendered possible by the use of titanium dioxides, which are known as UV light protection filters, moreover leads to a high UV light protection for the lips, which are particularly sensitive to solar radiation, being inherent in the formulations according to the invention.

### DETAILED DESCRIPTION OF THE PREFERRED EMBODIMENTS

The cosmetic or dermatological formulations according to the invention advantageously comprise a) a lipophilic phase in a concentration of 35 to 65% by weight, b) an aqueous phase in a concentration of 25 to 40% by weight, c) Pickering pigments from titanium dioxide having a particle diameter of 10 to 100 nm and/or iron oxides having a particle diameter of 100 to 800 nm in an amount of 1.5 to 4% by weight, d) coated titanium dioxide pigments having a particle diameter of 100 to 2,000 nm in an amount of 1 to 8% by weight and in particular 2 to 6% by weight, and e) further colored pigments in an amount of 2 to 15% by weight and in particular 5 to 10% by weight, in each case based on the total weight of the formulation.

A Pickering pigment from titanium dioxide which is advantageous according to the invention is, for example, Eusolex T 2000 (Merck).

Pickering pigments from iron oxides which are advantageous according to the invention are, for example, the synthetic iron oxides marketed by Bayer under the name Bayferrox (average particle diameter 0.1-0.8 μm).

Furthermore, it may be advantageous according to the inventions to add to the formulations according to the invention polymethylsilsesquioxanes as further Pickering pigments. For example, the polymethylsilsesquioxanes marketed by GE Bayer Silicones having an average particle diameter of 4-8 μm are advantageous according to the invention.

According to the invention, the lipophilic phase contained in the cosmetic or dermatological formulations according to the invention advantageously comprise a) waxes in a concentration of 1 to 25% by weight and particularly preferably 10 to 20% by weight and b) lipids in a concentration of 75 to 99% by weight and particularly preferably 80 to 90% by weight, in each case based on the total weight of the lipophilic phase.

Wax components which are advantageously to be used according to the invention can be chosen from the group consisting of plant waxes, animal waxes, mineral waxes and petrochemical waxes.

It is preferable if the wax component or the entirety of the wax components is chosen from the group consisting of esters of saturated or unsaturated, branched or unbranched alkanecarboxylic acids of a chain length of 1 to 80 C atoms and saturated or unsaturated, branched or unbranched alcohols of a chain length of 1 to 80 C atoms and from the group consisting of esters of aromatic carboxylic acids and saturated or unsaturated, branched or unbranched alcohols of a chain length of 1 to 80 C atoms, as long as the wax component or the entirety of the wax components are a solid at room temperature.

It is particularly preferable if the wax component or the entirety of the wax components is chosen from the group consisting of
esters of saturated branched alkanecarboxylic acids of a chain length of 14 to 44 C atoms and saturated branched alcohols of a chain length of 14 to 44 C atoms, as long as the wax component or the entirety of the wax components are a solid at room temperature,
naturally occurring waxes,
diesters of polyols or C10-C80 fatty acids,
ethoxylated waxes,
triglyceride waxes,
C16-C60 fatty acids (or salts thereof) or C16-C80 fatty alcohols.

Waxes which are favorable according to the invention are, for example, candelilla wax, carnauba wax, Japan wax, esparto grass wax, cork wax, guarama wax, rice germ oil wax, sugar cane wax, berry wax, ouricury wax, montan wax, jojoba wax, shea butter, beeswax, shellac wax, spermaceti, lanolin (wool wax), uropygium fat, ceresin, ozocerite (earth wax), paraffin waxes and microwaxes.

Further advantageous wax components are chemically modified waxes and synthetic waxes, such as, for example, those obtainable under the trade names Syncrowax HRC (glyceryl tribehenate), Syncrowax HGLC (C₁₆₋₃₆-fatty acid triglyceride) and Syncrowax AW 1C (C₁₈₋₃₆-fatty acid) from CRODA GmbH as well as montan ester waxes, Sasol waxes, hydrogenated jojoba waxes, polyalkylene waxes, polyethylene glycol waxes, and also chemically modified fats, such as e.g. hydrogenated plant oils (for example hydrogenated castor oil and hydrogenated coconut fatty glycerides), triglycerides, such as, for example, trihydroxystearin, fatty acids, fatty acid esters and glycol esters, such as, for example, C₂₀₋₄₀-alkyl stearate, C₂₀₄₀-alkyl hydroxystearoylstearate and glycol montanate.

The wax component or the entirety of the wax components of the formulations according to the invention is preferably chosen from the group consisting of esters of saturated or unsaturated, branched or unbranched alkanecarboxylic acids of a chain length of 14 to 44 C atoms and saturated or unsaturated, branched or unbranched alcohols of a chain length of 14 to 44 C atoms, from the group consisting of esters of aromatic carboxylic acids or hydroxycarboxylic acids (e.g. 12-hydroxystearic acid) and saturated or unsaturated, branched or unbranched alcohols of a chain length of 3 to 30 C atoms, as long as the wax component or the entirety of the wax components are a solid at room temperature.

It is preferable to choose the wax components from the group consisting of
esters of saturated branched alkanecarboxylic acids of a chain length of 14 to 44 C atoms and saturated unbranched alcohols of a chain length of 14 to 44 C atoms, as long as the wax component or the entirety of the wax components are a solid at room temperature,
and/or
esters of saturated unbranched alkanecarboxylic acids of a chain length of 14 to 44 C atoms and saturated branched alcohols of a chain length of 14 to 44 C atoms, as long as the wax component or the entirety of the wax components are a solid at room temperature.

It is particularly preferable to choose the wax components from the group consisting of esters of saturated branched alkanecarboxylic acids of a chain length of 14 to 44 C atoms and saturated branched alcohols of a chain length of 14 to 44 C atoms, as long as the wax component or the entirety of the wax components are a solid at room temperature.

The wax components can be particularly advantageously chosen from the group consisting of C₁₆₋₃₆-alkyl stearates, C₁₀₋₄₀-alkyl stearates, C₂₀₋₄₀-alkyl isostearates, C₂₀₋₄₀-dialkyl dimerates, C₁₈₋₃₈-alkyl hydroxystearoylstearates, C₂₀₋₄₀-alkyl erucates, and furthermore C₃₀₋₅₀-alkyl-beeswax, cetearyl behenate.

Those substances which have a melting point above 45° C., such as carnauba wax, candelilla wax, microcrystalline waxes and ozocerite, beeswax, synthetic waxes, such as polyethylene waxes or saturated esters of unbranched alkanecarboxylic acids, are very particularly preferably employed according to the invention as waxes.

The lipophilic phase furthermore can advantageously comprise lipids which are liquid at room temperature. These are lipids from the group consisting of branched and unbranched hydrocarbons, dialkyl ethers, the group consisting of saturated or unsaturated, branched or unbranched alcohols, and fatty acid triglycerides, namely the triglycerol esters of saturated or unsaturated, branched or unbranched alkanecarboxylic acids of a chain length of 8 to 24, in particular 12-18 C atoms.

The fatty acid triglycerides can advantageously be chosen, for example, from the group consisting of synthetic, semi-synthetic and naturally occurring oils, such as e.g. olive oil, sunflower oil, soya oil, groundnut oil, rape oil, almond oil, palm oil, coconut oil, castor oil, wheat germ oil, grape seed oil, thistle oil, evening primrose oil, macadamia nut oil and many of the like.

Coconut glycerides (Myritol 331), for example, are of importance according to the invention.

Lipids chosen from the group consisting of synthetic and naturally occurring esters of saturated or unsaturated, branched or unbranched alkanecarboxylic acids of a chain length of 3 to 30 C atoms and saturated or unsaturated, branched or unbranched alcohols of a chain length of 3 to 30 C atoms and from the group consisting of esters of aromatic carboxylic acids and saturated or unsaturated, branched or unbranched alcohols of a chain length of 3 to 30 C atoms, are furthermore preferred. Such ester oils can then advantageously be chosen from the group consisting of isopropyl myristate, isopropyl palmitate, isopropyl stearate, iso-propyl oleate, n-butyl stearate, n-hexyl laurate, n-decyl oleate, isooctyl stearate, iso-nonyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl ole-ate, erucyl erucate and synthetic, semi-synthetic and naturally occurring mixtures of such esters, such as e.g. jojoba oil.

Further oils according to the invention can be chosen from the group consisting of guerbet alcohols. The total amount of guerbet alcohols in the finished make-up formulation is advantageously chosen from the range up to 25.0 wt. %, preferably 0.5-15.0 wt. %, based on the total weight of the stick, octyidodecanol preferably being employed.

It is furthermore also advantageous to employ lipids from the group consisting of non-polar oils. These are, for example, those which are chosen from the group consisting of branched and unbranched hydrocarbons and hydrocarbon waxes, in particular mineral oil, Vaseline (petrolatum), paraffin oil, squalane and squalene, polyolefins and hydrogenated polyisobutenes. Among the polyolefins, polydecenes are the preferred substances.

The following Table 1 lists lipids which are advantageous according to the invention as individual substances or also in a mixture with one another. The relevant surface tensions against water are stated in the last column. However, it is also advantageous to use mixtures of higher and low-polar lipids and the like.

TABLE 1
Lipids which are advantageous according to the invention

| Trade name | INCI name | (mN/m) |
|---|---|---|
| Isofol ® 14 T | Butyl Decanol + Hexyl Decanol + Hexyl | 27.6 |
| | Octanol + Butyl Octanol | |
| Isofol ® 16 | Hexyl Decanol | 24.3 |
| Eutanol ® G | Octyldodecanol | 24.8 |
| Cetiol ® OE | Dicaprylyl Ether | 22.1 |
| Miglyol ® 812 | Caprylic/Capric Triglyceride | 21.3 |
| Cegesoft ® C24 | Octyl Palmitate | 23.1 |
| Isopropyl | Isopropyl Stearate | 21.9 |
| stearate | | |
| Estol ® 1540 | Octyl Octanoate | 30.0 |
| EHC | | |
| Finsolv ® TN | C₁₂₋₁₅ Alkyl Benzoate | 21.8 |
| Cetiol ® SN | Cetearyl Isononanoate | 28.6 |
| Dermofeel ® | Butylene Glycol Caprylate/Caprate | 21.5 |
| BGC | | |
| Trivent ® OCG | Tricaprylin | 20.2 |
| MOD | Octyldodeceyl Myristate | 22.1 |
| Cosmacol ® ETI | Di-C₁₂₋₁₃ Alkyl Tartrate | 29.4 |
| Miglyol ® 829 | Caprylic/Capric Diglyceryl Succinate | 29.5 |
| Prisorine ® 2036 | Octyl Isostearate | 29.7 |
| Tegosoft ® SH | Stearyl Heptanoate | 28.7 |
| Abil ® Wax 9840 | Cetyl Dimethicone | 25.1 |
| Cetiol ® LC | Coco-Caprylate/Caprate | 24.8 |
| IPP | Isopropyl Palmitate | 22.5 |
| Luvitol ® EHO | Cetearyl Octanoate | 28.6 |
| Cetiol ® 868 | Octyl Stearate | 28.4 |

The oily phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicaprylyl ether.

Mixtures of octyldodecanol, caprylic-capric acid triglyceride, dicaprylyl ether or mixtures of C₁₂₋₁₅-alky benzoate and 2-ethylhexyl isostearate, mixtures of C₁₂₋₁₅-alky benzoate and isotridecyl isononanoate as well as mixtures of C₁₂₋₁₅-alky benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene are advantageously to be used in the context of the present invention.

Any desired blends of oil and wax components can advantageously be employed in the context of the present invention.

It is of advantage to adjust the ratio of wax and lipid components to one another approximately from the range of weight ratios of between 1:20 to 2:1, in particular 1:5 to 4:3.

The aqueous phase of the cosmetic or dermatological formulations according to the invention advantageously comprises water in a concentration of 18 to 38% by weight as well as glycerol in a concentration of 2 to 10% by weight, in each case based on the total weight of the formulation.

The aqueous phase of the formulations according to the invention moreover optionally advantageously comprises
alcohols, diols or polyols of low C number, as well as ethers thereof, preferably ethanol, isopropanol, propylene glycol, ethylene glycol, ethylene glycol monoethyl or monobutyl ether, propylene glycol monomethyl, monoethyl or monobutyl ether, diethylene glycol monomethyl or monoethyl ether and analogous products, and furthermore alcohols of low C number, e.g. ethanol, isopropanol or water-soluble naturally occurring, naturally occurring modified or synthetic mono-, di- or polysaccharides, such as e.g. glucose, fructose, sucrose, sorbitol, xylitol or cyclodextrins, as well as, in particular, one or more thickeners, which can advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group consisting of polyacrylates, preferably a polyacrylate from the group consisting of so-called Carbopols, for example Carbopols of the types 980, 981, 1382, 2984, 5984, in each case individually or in combination.

The cosmetic or dermatological formulations according to the invention comprise a number of pigments. The dyestuffs and colored pigments can be chosen from the corresponding positive list of cosmetics legislation or the EC list of cosmetics coloring agents. In most cases they are identical to the dyestuffs approved for foodstuffs. Advantageous colored pigments are, for example, titanium dioxide, mica, iron oxides (e.g. Fe₂O₃, Fe₃O₄, FeO(OH)) and tin oxide. Advantageous dyestuffs are, for example, carmine, Berlin blue, chromium oxide green, ultramarine blue and manganese violet. It is particularly advantageous to choose the dyestuffs or colored pigments from the following list. The Color Index Numbers (CIN) can be found in the Rowe Colour Index, 3rd edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfonic acid diethylamide-1′- | 12490 | red |
| phenylazo)-3-hydroxy-5″-chloro-2″,4″-dimethoxy-2- | | |
| naphthoic acid anilide | | |
| Disperse Yellow 16 | 12700 | yellow |
| 1-(4-Sulfo-1-phenylazo)-4-aminobenzene-5-sulfonic acid | 13015 | yellow |
| 2,4-Dihydroxyazobenzene-4′-sulfonic acid | 14270 | orange |
| 2-(2,4-Dimethylphenylazo-5-sulfonic acid)-1- | 14700 | red |
| hydroxynaphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonic acid-4-chloro-5-carboxylic acid-1- | 15525 | red |
| phenylazo)-2-hydroxynaphthalene | | |
| 1-(3-Methylphenylazo-4-sulfonic acid)-2- | 15580 | red |
| hydroxynaphthalene | | |
| 1-(4′,(8′)-Sulfonic acid-naphthylazo)-2- | 15620 | red |
| hydroxynaphthalene | | |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic | 15850 | red |
| acid | | |
| 1-(2-Sulfo-4-methyl-5-chloro-1-phenylazo)-2-hydroxynaphthalene- | 15865 | red |
| 3-carboxylic acid | | |
| 1-(2-Sulfo-1-naphthylazo)-2-hydroxynaphthalene-3- | 15880 | red |
| carboxylic acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic | 16290 | red |
| acid | | |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxypyrazolone- | 19140 | yellow |
| 3-carboxylic acid | | |
| Pigment Yellow 16 | 20040 | yellow |
| 2,6-(4′-Sulfo-2″, 4″-dimethyl)bisphenylazo)1,3-dihydroxybenzene | 20170 | orange |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1- | 27755 | black |
| hydroxy-7-aminonaphthalene-3,6-disulfonic acid | | |
| 4′-[(4″-Sulfo-1″-phenylazo)-7′-sulfo-1′-naphthylazo]-1- | 28440 | black |
| hydroxy-8-acetyl-aminonaphthalene-3,5-disulfonic acid | | |
| Direct Orange 34, 39, 44, 46, 60 | 40215 | orange |
| Food Yellow | 40800 | orange |
| trans-β-Apo-8′-carotenealdehyde (C₃₀) | 40820 | orange |
| trans-Apo-8′-carotenic acid (C₃₀)-ethyl ester | 40825 | orange |
| Canthaxanthine | 40850 | orange |
| Acid Blue 1 | 42045 | blue |
| 2,4-Disulfo-5-hydroxy-4′-4″-bis(diethylamino)triphenylcarbinol | 42051 | blue |
| 4-[(-4-N-Ethyl-p-sulfobenzylamino)phenyl-(4-hydroxy-2- | 42053 | green |
| sulfophenyl)(methylene)-1-(N-ethylN-p-sulfobenzyl)-2,5- | | |
| cyclohexadieneimine] | | |
| Acid Blue 7 | 42080 | blue |
| (N-Ethyl-p-sulfobenzylamino)phenyl-(2-sulfophenyl)- | 42090 | blue |
| methylene-(N-ethyl-N-p-sulfobenzyl)Δ^{2,5}- | | |
| cyclohexadieneimine | | |
| Acid Green 9 | 42100 | green |
| Diethyldisulfobenzyl-di-4-amino-2-chloro-di-2-methylfuchsoneimmonium | 42170 | green |
| Basic Violet 14 | 42510 | violet |
| Basic Violet 2 | 42520 | violet |
| 2′-Methyl-4′-(N-ethyl-N-m-sulfobenzyl)amino-4″-(N- | 42735 | blue |
| diethyl)-amino-2-methyl-N-ethylN-m- | | |
| sulfobenzylfuchsoneimmonium | | |
| 4′-(N-Dimethyl)amino-4″-(N-phenyl)aminonaphtho-N- | 44045 | blue |
| dimethylfuchsoneimmonium | | |
| 2-Hydroxy-3,6-disulfo-4,4′-bisdimethylaminonaphthofuchsoneimmonium | 44090 | green |
| Acid Red 52 | 45100 | red |
| 3-(2′-Methylphenylamino)-6-(2′-methyl-4′-sulfo- | 45190 | violet |
| phenylamino)-9-(2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |

It may furthermore be favorable to choose as the dyestuff one or more substances from the following group: 2,4-dihydroxyazobenzene, 1-(2′-chloro-4′-5 nitro-1′-phenylazo)-2-hydroxynaphthalene, Ceres Red, 2-(4-sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naph-thylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 8-amino-2-phenylazo-1-naphthol-3,6-disulfonic acid, aluminum salt of 4-(4-sulfo-1-phenyl-azo)-1-(4-sulfophenyl)-5-hydroxypyrazolone-3-carboxylic acid, 4′-[(4″-sulfo-1″-phenylazo)-7′-sulfo-1′-naphthylazo]-1-hydroxy-8-acetylaminonaphthalene-3,5-disulfonic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalonedisulfonic acid, aluminum salt of indigodisulfonic acid, 4,4′-dimethyl-6,6′-dichlorothioindigo, complex salt (Na, Al, Ca) of carminic acid, red and black iron oxide (CIN: 77 491 (red) and 77 499 (black)), iron oxide hydrate (CIN: 77 492), manganese ammonium diphosphate (CIN 77745), ultramarine (CIN 77007) and titanium dioxide.

Formulations according to the invention comprise titanium dioxides which can be both in the rutile crystal modification and in the anatase form and in the context of the present invention can advantageously be treated on the surface (“coated”), whereby, for example, a hydrophilic, amphiphilic or hydrophobic character is to be formed or retained. This surface treatment can comprise providing the pigments with a thin hydrophilic or hydrophobic inorganic or organic layer by processes known per se. The various surface coatings can also comprise water in the context of the present invention.

Inorganic surface coatings in the context of the present invention can comprise aluminum oxide (Al₂O₃), aluminum hydroxide Al(OH)₃, or aluminum oxide hydrate (also: alumina, CAS no.: 1333-84-2), sodium hexametaphosphate (NaPO₃)₆, sodium metaphosphate (NaPO₃)ₙ, silicon dioxide (SiO₂) (also: silica, CAS no.: 7631-86-9), zirconium oxide (ZrO₂) or iron oxide (Fe₂O₃). These inorganic surface coatings can occur by themselves, in combination, or in combination with organic coating materials.

For this purpose, oxides, oxide hydrates or phosphates, for example of the elements Al, Si, Zr, are precipitated on the pigment surface in dense layers. The inorganic after-treatment is in general effected in an aqueous suspension of the pigment by addition of soluble after-treatment chemicals, such as e.g. aluminum sulfate, and subsequent precipitation of the hydroxide, which is poorly soluble in the neutral range, by controlled adjustment of the pH with sodium hydroxide solution. After the inorganic after-treatment, the coated pigments are separated off from the suspension by filtration and washed thoroughly, in order to remove the dissolved salts, and the pigments isolated are then dried.

Titanium dioxides on to the surface of which aluminum hydroxide has been applied, such as, e.g., the titanium dioxide types C47-051 and C47-5175 obtainable from Sun Chemical, are particularly preferred in the context of this invention. Pigments which are furthermore preferred are titanium dioxides which are coated with aluminum or silicon oxides, such as e.g. from Krosnos Titan: Kronos 1071 and 1075 or from Kingfisher: A310.03 Tudor Aspen.

Organic surface coatings in the context of the present invention can comprise plant or animal aluminum stearate, plant or animal stearic acid, lauric acid, dimethylpolysiloxane (also: dimethicone), methylpolysiloxane (methicone), simethicone (a mixture of dimethylpolysiloxane with an average chain length of 200 to 350 dimethylsiloxane units and silica gel) or algic acid. These organic surface coatings can occur by themselves, in combination, or in combination with inorganic coating materials.

It may furthermore be advantageous according to the invention to employ pearlescent pigments. These include naturally occurring pearlescent pigments, such as, e.g.,
“fish silver” (guanine/hypoxanthine mixed crystals from fish scales) and
“mother-of-pearl” (ground mussel shells),
monocrystalline pearlescent pigments, such as e.g. bismuth oxychloride (BiOCl), and
laminar substrate pigments: e.g. mica/metal oxide

Pulverulent pigments or castor oil dispersions of bismuth oxychloride or titanium dioxide as well as bismuth oxychloride or titanium dioxide on mica form the basis, for example, of pearlescent pigments. The lustrous pigment listed under CIN 77163 is e.g. particularly advantageous.

The following pearlescent pigment types based on mica/metal oxide are, for example, furthermore advantageous:

| | Coating/ | |
|---|---|---|
| Group | layer thickness | Color |
| Silver-white | TiO₂: 40-60 nm | silver |
| pearlescent pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Colored lustrous | Fe₂O₃ | bronze |
| pigments | | |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | golden |
| | | shades |
| | TiO₂/Cr₂O₃ | green |
| | TiO₂/Berlin blue | deep blue |
| | TiO₂/carmine | red |

The pearlescent pigments obtainable from Merck under the trade names Timiron, Colorona or Dichrona are particularly preferred.

The list of pearlescent pigments mentioned of course is not intended to be limiting. Pearlescent pigments which are advantageous in the context of the present invention are obtainable by numerous routes which are known per se. For example, other substrates apart from mica can also be coated with further metal oxides, such as silica and many of the like. SiO₂ particles coated with e.g. TiO₂ and Fe₂O₃ (“Ronaspheres”) which are marketed by Merck are advantageous.

It may moreover be advantageous to dispense with a substrate, such as mica, entirely. Pearlescent pigments which are prepared using SiO₂ are particularly preferred. Such pigments, which can also additionally have gonichromatic effects, are obtainable, e.g., from BASF under the trade name Sicopearl Fantastico.

Pigments from Engelhard/Mearl which are based on calcium sodium borosilicate and are coated with titanium dioxide can furthermore advantageously be employed. These are obtainable under the name Reflecks. Due to their particle size of 40-180 μm, they have a glitter effect, in addition to the color.

The dyestuffs and pigments can be present both individually and in a mixture, and can also be mutually coated with one another, various color effects in general being brought about by different coating thicknesses. The total amount of dyestuffs and coloring pigments is advantageously chosen from the range of e.g. 0.1 wt. % to 30 wt. %, preferably 0.5 to 15 wt. %, in particular 1.0 to 10 wt. %, in each case based on the total weight of the formulations.

The invention also relates to the process of incorporation of colored pigments into the formulations according to the invention. In this process, the colored pigments are first predispersed in a cosmetics oil, before this dispersion is incorporated into the formulation. Oils which are advantageous according to the invention for the preparation of the dispersion of the dyestuffs are, above all, polar oils, in particular castor oil, octyidodecanol, lanolin oil or diisostearyl fumarate.

It may furthermore be of advantage to modify the sensory properties of the stick by addition of so-called fillers. Fillers in the context of the present invention are particulate substances, which as a rule generate no color effect in the cosmetic formulation in which they are employed. Fillers according to the invention furthermore conventionally have a low refractive index and no or only a very low covering power resulting therefrom.

A number of fillers which serve, e.g., as carrier materials in the formulation of powders or as viscosity and sensory modulators in emulsions or anhydrous formulations are known from the prior art. Such fillers are often also employed in order to acquire matting effects on the skin or to absorb sebum.

Moreover, the use of fillers in general also influences the ease of distribution of conventional formulations on the skin and the uniformity of a possible color effect.

Suitable fillers in the context of the invention are boron nitride, nylon 12, polymethylsilsesquioxanes, polymethyl methycrylates, lauroyllysines or bismuth oxychloride.

The cosmetic or dermatological formulations according to the invention can comprise cosmetics auxiliaries such as are conventionally used in such formulations, e.g., perfumes, UV filters, thickeners, preservatives, or other conventional constituents of a cosmetic or dermatological formulation, such as alcohols, polyols, polymers, electrolytes, organic solvents or silicone derivatives.

An additional content of antioxidants is in general preferred. According to the invention, all the antioxidants which are suitable or usual for cosmetic or dermatological uses can be used as favorable antioxidants.

The antioxidants are advantageously chosen from the group consisting of amino acids (e.g. glycine, histidine, tyrosine, tryptophan) and derivatives thereof, imidazoles (e.g. urocaninic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, liponic acid and derivatives thereof (e.g. dihydroliponic acid), aurothioglucose, propylthiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, y-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) as well as sulfoximine compounds (e.g. buthionine sulfoximines, homocysteine sulfoximine, buthionine sulfones, penta-, hexa-, heptathionine sulfoximine) in very small tolerable dosages (e.g. pmol to μmol/kg), furthermore (metal) chelating agents (e.g. α-hydroxy-fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. y-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutic acid and derivatives thereof, α-glycosylrutin, ferulic acid, furfurylideneglucitol, carnosine, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄) selenium and derivatives thereof (e.g. selenium-methionine), stilbenes and derivatives thereof (e.g. stilbene oxide, trans-stilbene oxide) and the derivatives (salts, esters, ethers, sugars, nucleotides, nucleosides, peptides and lipids), which are suitable according to the invention, of these active compounds mentioned.

The amount of the abovementioned antioxidants (one or more compounds) in the formulations is preferably 0.001 to 30 wt. %, particularly preferably 0.05-20 wt. %, in particular 0.1-10 wt. %, based on the total weight of the formulation.

If vitamin E or derivatives thereof is/are the antioxidant(s), it is advantageous to choose the particular concentration thereof from the range of 0.001-10 wt. %, based on the total weight of the formulation.

If vitamin A or vitamin A derivatives or carotenes or derivatives thereof is/are the antioxidant(s), it is advantageous to choose the particular concentrations thereof from the range of 0.001-10 wt. %, based on the total weight of the formulation.

According to the invention, preservatives are furthermore to be added to the formulation according to the invention. Preservatives which are approved in foodstuffs technology and are listed below with their E numbers are advantageously to be used according to the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na salt |
| E 216 | n-Propyl p-hydroxybenzoate |
| E 217 | n-Propyl p-hydroxybenzoate Na |
| | salt |
| E 218 | Methyl p-hydroxybenzoate |
| E 219 | Methyl p-hydroxybenzoate Na |
| | salt |
| E 220 | Sulfur dioxide |
| E 221 | Sodium sulfite |
| E 222 | Sodium hydrogen sulfite |
| E 223 | Sodium disulfite |
| E 224 | Potassium disulfite |
| E 226 | Calcium sulfite |
| E 227 | Calcium hydrogen sulfite |
| E 228 | Potassium hydrogen |
| | sulfite) |
| E 230 | Biphenyl (Diphenyl) |
| E 231 | Orthophenylphenol |
| E 232 | Sodium |
| | orthophenylphenolate |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

According to the invention, preservatives or preservative auxiliaries which are usual in the cosmetics field dibromodicyanobutane (2-bromo-2-bromomethylglutarodi-nitrile), 3-iodo-2-propynyl butyl carbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol are furthermore suitable. Agents which split off formaldehyde.

According to the invention, these are preservatives or preservative auxiliaries which are usual in the cosmetics field and such as are also listed in cosmetics legislation. Those which are particularly preferably employed are 3-iodo-2-propynyl-butyl carbamateimidazolidinylurea, diazolinidylurea (e.g. obtainable from ISP Sutton Laboratories under the trade name Germall II), 5-chloro-2-methyl-4-isothiazolin-3-one and 2-methyl-4-isothiazolone, which are marketed as a mixture under the trade names Kathon CG and Rokonsal S1,1,3-dimethyloyl-5,5-dimethylhydantoin, which is marketed by itself under the name Glydant by Lonza or in a mixture with 3-iodo-2-propynyl butyl carbamate under the name Glydant Plus, 2-Phenyl hydroxyalkyl ethers, in particular the compound known under the name phenoxyethanol, are furthermore suitable as preservatives on the basis of their bactericidal and fungicidal actions on a number of microorganisms. Silver chloride, which is marketed e.g. by the company Johnson Matthey as a mixture with titanium dioxide under the name J M Acticare, is furthermore also preferred.

Other germ-inhibiting agents are also likewise suitable for incorporation into the formulations according to the invention. Advantageous substances are, for example, 2,4,4′-trichloro-2′-hydroxydiphenyl ether (Irgasan), 1,6-di-(4-chlorophenylbiguanido)-hexane (chlorhexidine), 3,4,4′-trichlorocarbanilide, quaternary ammonium compounds, clove oil, mint oil, thyme oil, triethyl citrate, farnesol (3,7,11-trimethyl-2,6,10-dodecatrien-1-ol) as well as the active compounds and active compound combinations described in the laid-open patent specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium bicarbonate is also advantageously to be used.

The cosmetic or dermatological formulations according to the invention are advantageously supplied in a lipstick casing. For this, the lipstick composition is cast in a mold and, after cooling, the casting is placed in the pan of the casing. Alternatively, the stick can also be cast directly in a special packing, so-called direct filling. It is essential for the stability of the stick in the casing that this casing is made of waterproof material or is coated with waterproof material and has a corresponding tightness.

Needless to say, the invention also relates to a lipstick casing containing a cosmetic or dermatological formulation according to the invention.

The invention furthermore relates to the use of the cosmetic and/or dermatological formulation as a lipstick, in particular as a decorative lipstick.

The invention furthermore relates to the use of the cosmetic and/or dermatological formulation according to the invention as UV light protection for the lips.

The following examples are intended to illustrate the present invention without limiting it. Unless stated otherwise, all the amounts data, contents and percentage contents are based on the weight and the total amount or on the total weight of the formulations.

### RECIPE EXAMPLES

| | 1 | 2 | 3 | 4 | 5 | 6 | 7 |
|---|---|---|---|---|---|---|---|
| Microcrystalline wax | 4.5 | 4 | 2.6 | 1.5 | 4.0 | 2.6 | 3.0 |
| Ozocerite | | 0.5 | 0.5 | 0.5 | | | 0.5 |
| Carnauba wax | 1.5 | 2.1 | 1.8 | 1.8 | 2.0 | 3.2 | 1.5 |
| Candelilla wax | 4.0 | 2.0 | 2.0 | 6.0 | 2.0 | 3.0 | 2.5 |
| Beeswax | | 1.0 | | | 1.0 | 0.6 | 0.5 |
| C24-40 Alkyl Stearate | | | 2.0 | 1.5 | | 3.5 | 1.5 |
| Lauryl Pyrrolidonecarboxylic | | 3.0 | | | | | 1.0 |
| Acid | | | | | | | |
| Lanolin oil | 4.0 | | | 2.0 | | | 3.0 |
| Bisdiglyceryl Polyacyladipate-2 | 3.5 | 5.0 | | | 6.0 | | 3.0 |
| Simethicone | 1.0 | | 0.5 | | | | 0.5 |
| Isopropyl Palmitate | 3.5 | 4.0 | | 2.0 | | 8.0 | 2.0 |
| Triisostearin | 3.0 | | | | | 2.0 | 2.0 |
| Myristyl Lactate | 4.0 | | 2.0 | 4.0 | | | 1.0 |
| Jojoba oil | 2.0 | | | 3.0 | 3.0 | | |
| Diisostearyl Malate | | | | 1.2 | | | 0.7 |
| Caprylic/Capric Triglyceride | | 5.0 | 3.0 | | | 3.0 | 2.0 |
| Pentaerythrityl | | | 2.0 | | 3.0 | | 2.0 |
| Tetraisostearate | | | | | | | |
| Isodecyl Neopentanoate | | 2.0 | | | 2.0 | | |
| Dicaprylyl Carbonate | | 2.0 | 2.0 | 3.0 | 4.0 | | |
| C12-15 Alkyl Benzoate | | | | 8.0 | | | |
| Hydrogenated Polydecene | 2.5 | | | | 8.0 | 6.0 | 3.0 |
| Squalane | | | 2.0 | | | | |
| Octyldodecanol | 2.5 | | to | 3.0 | | 4.0 | 3.0 |
| | | | 100 | | | | |
| Diisostearyl Fumarate | | | | to | | | |
| | | | | 100 | | | |
| PVP/Hexadecene Copolymer | | 0.5 | | | | 0.3 | 0.5 |
| Lecithin | | | | | | 0.5 | |
| Ethylhexyl Methoxycinnamate | | 0.5 | | 2.0 | 0.5 | 3.5 | |
| Butyl | | 0.2 | | | 0.2 | | |
| Methoxydibenzoylmethane | | | | | | | |
| Bisethylhexyloxyphenol Methoxyphenyl | | | | 0.25 | | 0.25 | 0.25 |
| Triazine | | | | | | | |
| Micronized titanium dioxide | 2.0 | 2.0 | 3.0 | | | | 1.5 |
| (Eusolex T 2000) | | | | | | | |
| Silica Dimethyl Silylate | | 0.3 | | | | 0.2 | |
| (Aerosil R972) | | | | | | | |
| Polymethylsilsesquioxane | | | 1.0 | 3.0 | | | 1.0 |
| (Tospearl 2000B) | | | | | | | |
| Bayferrox 1120 Z (red) | | | | | 4.0 | 3.2 | 0.5 |
| Bayferrox 315 Z (black) | | | | | | 0.6 | 0.2 |
| Bayferrox 920 Z (yellow) | | | | | | 0.2 | 0.2 |
| Interference pigments | | 4.0 | 3.0 | | | | 2.0 |
| Titanium dioxide CI 77891 | 4.0 | 3.8 | 2.0 | 3.0 | | 0.5 | 1.0 |
| Iron oxides CI 77491, 77492, | 3.2 | 2.2 | | 2.2 | | | 2.5 |
| 77499 | | | | | | | |
| D&C Red 7 | 0.6 | | 1.9 | 1.3 | 4.2 | | 0.5 |
| D&C Red 6 | | | 0.4 | | | 3.2 | 1.2 |
| FD&C Blue 1 | | | | 0.3 | 0.3 | | |
| D&C Red 33 | | 0.3 | 2.2 | | | | 0.8 |
| D&C Red 21 | | | | 0.5 | | | |
| FD&C Yellow 6 | | 1.5 | | | | 0.8 | |
| D&C Red 30 | | | | | 0.2 | | 0.8 |
| D&C Red 28 | | | | | | 0.5 | 0.5 |
| D&C Red 34 | | | 0.3 | 0.8 | | | 0.2 |
| Tocopheryl Acetate | 1.0 | | 1.5 | | 1.0 | 2.0 | 1.0 |
| Ubiquinone | | | 0.1 | | | 0.05 | |
| Xylitol | 2.0 | | 2.0 | 2.0 | | | 2.0 |
| Glycerol | 5.0 | 3.0 | 3.0 | 5.0 | 3.0 | 10.0 | 3.0 |
| Preservative, BHT, perfume, | q.s. | q.s. | q.s. | q.s. | q.s. | q.s. | q.s. |
| aroma | | | | | | | |
| Water | 30.0 | 40.0 | 50.0 | 20.0 | 30.0 | 30.0 | 40.0 |
| Castor oil | to 100 | to 100 | 5.0 | | to 100 | to 100 | to 100 |

1. A cosmetic or dermatological formulation comprising
(a) a lipophilic phase in a concentration of 20 to 70% by weight,
(b) an aqueous phase in a concentration of 20 to 40% by weight,
(c) at least one Pickering pigment selected from the group consisting of titanium dioxide pigments having a particle diameter of 10 to 100 nm and iron oxide pigments having a particle diameter of 100 to 800 nm, said at least one Pickering pigment present in a concentration of 1 to 6% by weight,
(d) at least one coated titanium dioxide pigment having a particle diameter of 100 to 2000 nm, in a concentration of 0.1 to 10% by weight,
(e) further colored pigments in a concentration of 0.1 to 20% by weight,
wherein the % by weight is in each case based on the total weight of the formulation.

2. The cosmetic or dermatological formulation as claimed in claim 1, wherein the lipophilic phase comprises waxes in a concentration of 1 to 25% by weight and lipids in a concentration of 75 to 99% by weight, in each case based on the total weight of the lipophilic phase.

3. The cosmetic or dermatological formulation as claimed in claim 1, wherein the lipophilic phase comprises at least one wax selected from the group consisting of microcrystalline waxes, carnauba wax, and candelilla wax.

4. The cosmetic or dermatological formulation as claimed in claim 1, wherein the aqueous phase comprises water in a concentration of 18 to 38% by weight and glycerol in a concentration of 2 to 10% by weight, in each case based on the total weight of the formulation.

5. The cosmetic or dermatological formulation as claimed in claim 1, further comprising a polymethylsilsesquioxane Pickering pigment having an average particle diameter of 4-8 μm.

6. the cosmetic or dermatological formulation as claimed in claim 1, wherein the at least one coated titanium dioxide pigment includes titanium dioxide coated with aluminum hydroxide.

7. The cosmetic or dermatological formulation as claimed in claim 1, wherein the formulation is a lipstick.

8. A lipstick casing containing a cosmetic or dermatological formulation as claimed in claim 1.

9. A method of manufacturing lipstick comprising combining into a formulation
(a) a lipophilic phase in a concentration of 20 to 70% by weight,
(b) an aqueous phase in a concentration of 20 to 40% by weight,
(c) at least one Pickering pigment selected from the group consisting of titanium dioxide pigments having a particle diameter of 10 to 100 nm and iron oxide pigments having a particle diameter of 100 to 800 nm, said at least one Pickering pigment present in a concentration of 1 to 6% by weight,
(d) at least one coated titanium dioxide pigment having a particle diameter of 100 to 2000 nm, in a concentration of 0.1 to 10% by weight,
(e) further colored pigments in a concentration of 0.1 to 20% by weight,
wherein the % by weight is in each case based on the total weight of the formulation.

10. The method as claimed in claim 9, further comprising the step of predispersing the further colored pigments in an oil prior to said combining step.

11. The method as claimed in claim 9, wherein the lipophilic phase comprises waxes in a concentration of 1 to 25% by weight and lipids in a concentration of 75 to 99% by weight, in each case based on the total weight of the lipophilic phase.

12. The method as claimed in claim 9, wherein the lipophilic phase comprises at least one wax selected from the group consisting of microcrystalline waxes, carnauba wax, and candelilla wax.

13. The method as claimed in claim 9, wherein the aqueous phase comprises water in a concentration of 18 to 38% by weight and glycerol in a concentration of 2 to 10% by weight, in each case based on the total weight of the formulation.

14. The method as claimed in claim 9, further comprising a polymethylsilsesquioxane Pickering pigment having an average particle diameter of 4-8 μm.

15. The method as claimed in claim 9, wherein the at least one coated titanium dioxide pigment includes titanium dioxide coated with aluminum hydroxide.

16. The method as claimed in claim 9, further comprising depositing the formulation into a lipstick casing.

17. A method of protecting the lips from UV light comprising applying to the lips a cosmetic or dermatological formulation comprising
(a) a lipophilic phase in a concentration of 20 to 70% by weight,
(b) an aqueous phase in a concentration of 20 to 40% by weight,
(c) at least one Pickering pigment selected from the group consisting of titanium dioxide pigments having a particle diameter of 10 to 100 nm and iron oxide pigments having a particle diameter of 100 to 800 nm, said at least one Pickering pigment present in a concentration of 1 to 6% by weight,
(d) at least one coated titanium dioxide pigment having a particle diameter of 100 to 2000 nm, in a concentration of 0.1 to 10% by weight,
(e) further colored pigments in a concentration of 0.1 to 20% by weight,
wherein the % by weight is in each case based on the total weight of the formulation.
